(19) United States
(12) Patent Application Publication
(10) Pub. No.: US 2015/0313560 A1
(43) Pub. Date: Nov. 5, 2015

(54) RADIATION GENERATION APPARATUS AND RADIOGRAPHIC IMAGING SYSTEM

(71) Applicant: CANON KABUSHIKI KAISHA, Tokyo (JP)

(72) Inventor: Satoru Omura, Chigasaki-shi (JP)

(21) Appl. No.: 14/701,332

(22) Filed: Apr. 30, 2015

(30) Foreign Application Priority Data
May 2, 2014 (JP) 2014-095130

(51) Int. Cl. A61B 6/00

(52) U.S. Cl. CPC A61B 6/4441; A61B 6/4405; A61B 6/54; A61B 6/542

(57) ABSTRACT

A radiographic imaging system and a radiation generation apparatus are provided where components are detachable according to a transportation form. The radiation generation apparatus and the radiographic imaging system include a radiation generation unit configured to generate radiation, an arm configured to support the radiation generation unit, a support pillar configured to support the arm, and a storage unit configured to store a radiation detector configured to detect the radiation. The storage unit is detachable from the support pillar.

### BACKGROUND OF THE INVENTION

1. Field of the Invention

The present invention relates to a radiation generation apparatus including a radiation generation unit configured to generate radiation for imaging a subject, and a radiographic imaging system.

2. Description of the Related Art

Portable radiation generation apparatus and portable radiographic imaging systems have been developed and are well known. When carrying out imaging by using a portable radiation generation apparatus, a radiation generation unit is installed according to a part of a subject to be imaged. Radiation applied from the radiation generation unit is detected by a radiation detector.

U.S. Pat. No. 6,754,306 discloses a portable radiography assembly including a radiation generation apparatus and a radiation detector; the assembly can be stored in box. However, when a radiation generation apparatus is moved via a moving portion including a plurality of wheels, the radiation generation apparatus and the radiation detector for detecting radiation must be carried separately as they have separate device bodies.

In the case of a technology discussed in Japanese Patent Application Laid-Open No. 2007-144161, a radiation generation apparatus includes a storage unit configured to store a radiation detector for detecting radiation. However, since a component such as the storage unit cannot be separated, it is difficult to achieve weight reduction of the radiation generation apparatus. Therefore, it is difficult to maneuver the radiation generation apparatus while lifting or moving the apparatus.

### SUMMARY OF THE INVENTION

The present invention is directed to a radiation generation apparatus configured such that a component is detachable according to a transportation form, and a radiographic imaging system.

According to aspects of the present invention, a radiation generation apparatus and a radiographic imaging system are disclosed. The radiation generation apparatus and the radiographic imaging system each includes a radiation generation unit configured to generate radiation, an arm configured to support the radiation generation unit, a support pillar configured to support the arm, a moving portion configured to support the support pillar and be movable, and a storage unit attachable to the support pillar and configured to store a radiation detector configured to detect the radiation. The storage unit is detachable from the support pillar. According to the radiation generation apparatus and the radiographic imaging system, components can be detached according to a transportation form, and thus transportability of the apparatus and/or system is improved.

Further features of the present invention will become apparent from the following description of exemplary embodiments with reference to the attached drawings.

### BRIEF DESCRIPTION OF THE DRAWINGS

FIG. 1 is a diagram illustrating a configuration of a radiation generation apparatus according to a first exemplary embodiment of the present invention.

FIG. 2 is a diagram illustrating a separation form (transportation form) of the radiation generation apparatus according to the first exemplary embodiment of the present invention.

FIGS. 3A and 3B are diagrams each illustrating a detaching mechanism of a storage unit of the radiation generation apparatus according to the first exemplary embodiment of the present invention.

FIGS. 4A and 4B are diagrams each illustrating a detaching mechanism of a control unit of the radiation generation apparatus according to the first exemplary embodiment of the present invention.

FIG. 5 is a diagram illustrating another view of the detaching mechanism of the control unit of the radiation generation apparatus according to the first exemplary embodiment of the present invention.

FIGS. 6A and 6B are diagrams each illustrating positions of centers of gravity of the storage unit and the control unit of the radiation generation apparatus according to the first exemplary embodiment of the present invention.

FIG. 7 is a diagram illustrating a configuration of a radiation generation apparatus according to a second exemplary embodiment of the present invention.

FIG. 8 is a diagram illustrating a configuration of a radiation generation apparatus according to a third exemplary embodiment of the present invention.

FIGS. 9A and 9B are diagrams each illustrating a configuration and operation of the radiation generation apparatus according to the third exemplary embodiment of the present invention.

### DESCRIPTION OF THE EMBODIMENTS

Hereinafter, the exemplary embodiments of the present invention will be described with reference to the accompanying drawings.

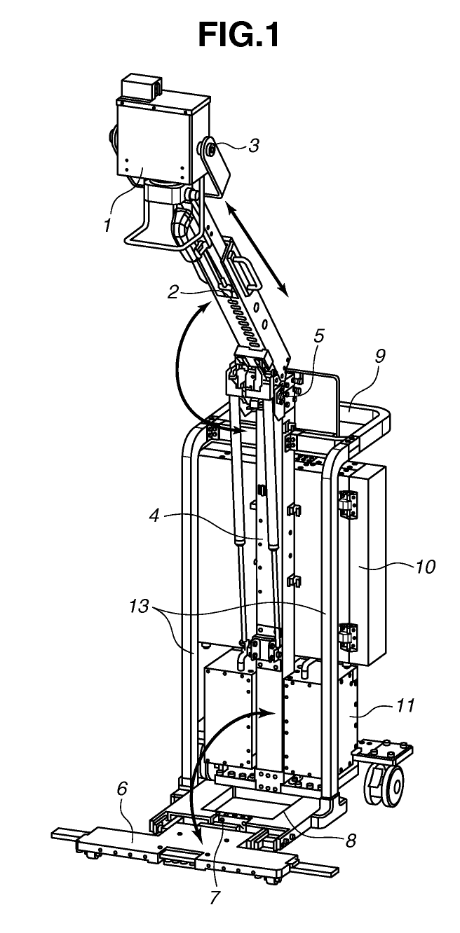

Hereinafter, a first exemplary embodiment will be described. FIG. 1 is a diagram illustrating a configuration of a radiation generation apparatus according to the present invention. The radiation generation apparatus mainly includes a moving portion 8 (moving or movable base) having a plurality of wheels installed therein, a support pillar 4 erected vertically on the moving potion 8, and an arm 2 installed to be movable with respect to (rotatable around) the support pillar 4. The radiation generation apparatus further includes a radiation generation unit 1 installed at the distal end of the arm and configured to be moveable with respect to (rotatable around) the arm 2; the radiation generation unit is configured to generate radiation to irradiate a subject. The radiation generation apparatus further includes a leg portion 6 foldable with respect to the moving portion 8.

The radiation generation unit 1 configured to generate radiation includes a radiation tube for irradiating radiation and a diaphragm for limiting a radiation irradiation area. The radiation generation unit is, for example, an X-ray transmissive-type radiation generation unit configured to generate X-ray radiation. The radiation generation unit 1 can also be implemented as a rotating-anode-type radiation generation unit. However, in the transmissive-type radiation generation unit, it is not necessary to cover a radiation generation tube or an envelope for housing the radiation generation tube all around with a shielding member made of lead or the like. Thus, the transmissive-type radiation generation unit can be reduced more in size and weight than a rotating anode radiation generation unit.

The arm 2 supports the radiation generation unit 1. The arm 2 has one end (distal end) connected to the radiation generation unit 1 and the other end (proximal end) connected to the support pillar 4. The arm 2 supports the radiation generation unit 1 and has a predetermined length. The arm 2 has an extension mechanism by which it can extend/contract in its longitudinal direction. The arm is a telescopic type. By extending the arm 2 to a predetermined length, the radiation generation unit 1 can be protruded to a subject side.

A rotor 3 is installed between the radiation generation unit 1 and the arm 2 (at the distal end of the arm 2), and configured to rotate the radiation generation unit 1. By rotating the radiation generation unit 1, radiation can be applied in a desired direction.

The support pillar 4 supports the arm 2. In an erected state of the radiation generation apparatus, the support pillar 4 is set at an angle vertical or nearly vertical to a horizontal surface (floor surface or ground surface). The support pillar 4 is installed at the moving portion 8. The support pillar 4 is erected on the moving portion 8 in a vertical direction. The support pillar 4 has a predetermined length, and may have an extension mechanism by which it can extend/contract in its longitudinal direction.

An arm hinge portion 5 connects the arm 2 to the support pillar 4 and makes the arm 2 openable/closable to the support pillar 4. The arm hinge portion 5 has a mechanism for connecting the arm 2 to the support pillar 4 and making the arm 2 openable/closable to the support pillar 4. The arm 2 can rotate around an upper end of the support pillar 4. The arm 2 can be folded to be substantially parallel to the support pillar 4. The arm 2 is rotated within a predetermined range (about 180°) in a predetermined rotating direction. The arm 2 is folded to a side opposite to a side where a handle 9 described below, a storage unit 10 configured to store the radiation detector, and a control unit 11 configured to control exposure of the radiation generation unit 1 are installed.

The leg portion 6 is a member for supporting the radiation generation apparatus. In the form illustrated in FIG. 1, in the radiation generation apparatus, components contacting the horizontal surface (floor surface or ground surface) are the moving portion 8 and the leg portion 6. In other words, the moving portion 8 and the leg portion 6 support the radiation generation apparatus. The leg portion 6 can increase an area of contact with the horizontal surface in the radiation generation apparatus. Thus, for example, even when the radiation generation unit 1 is positioned with respect to the subject, weight balance of the radiation generation apparatus can be maintained by the leg portion 6.

Specifically, the leg portion 6 is a plate member, and includes a support portion configured to support the radiation generation apparatus while contacting the horizontal surface (floor surface or ground surface). The support portion is installed at a bottom surface of the leg portion 6. The support portion may be a moving mechanism such as a plurality of tires or casters which move on the horizontal surface (floor surface or ground surface). The leg portion 6 is foldable in a predetermined rotating direction with respect to the moving portion 8. The leg portion 6 is rotated within a range of about 90° in the predetermined rotating direction. A rotational axis of the leg portion 6 is parallel to that of the arm 2.

The leg portion 6 is connected to the moving portion 8 via a support leg hinge portion 7. The leg portion 6 can be folded with the support leg hinge portion 7. During photographing, as illustrated in FIG. 1, the leg portion 6 is unfolded by the operator, and the radiation generation apparatus is supported by the leg portion 6 and the moving portion 8. At this time, the support leg hinge portion 7 may control rotation of a hinge to fix the leg portion 6 to the moving portion 8.

The handle 9 is gripped by the operator when moving the radiation generation apparatus in a horizontal direction. As illustrated in FIG. 1, the handle 9 is installed at the upper end of the support pillar 4. The operator can move the moving portion 8 by gripping the handle 9 and pushing the handle 9 in an advancing direction of the moving portion 8.

When the radiation generation apparatus needs to be lifted and carried, the operator first folds the arm 2 to store the arm 2 and the radiation generation unit 1, and closes the leg portion 6. The leg portion 6 serves as a cover for the radiation generation unit 1 so that the radiation generation unit 1 can be protected. In the state where the leg portion 6 and the arm 2 are folded, all the components of the radiation generation apparatus are accordingly arranged on the moving portion 8.

Further, in the state where the leg portion 6 and the arm 2 are folded, heavy articles such as the radiation generation unit 1 and the control unit 11 are arranged near the horizontal surface (floor surface or ground surface), and thus in the radiation generation apparatus, a center of gravity can be placed at a low position. In other words, the radiation generation unit 1 is disposed nearer to the horizontal surface (floor surface or ground surface) than an upper end of the folded leg portion 6. Therefore, the weight balance of the radiation generation apparatus can be maintained.

The moving portion 8 includes a plurality of wheels. The wheels are a plurality of tires or casters, and always placed on the horizontal surface (floor surface or ground surface). By rotating the wheels, the radiation generation apparatus can be moved back and forth together with the moving portion 8. The moving portion 8 can be replaced by a base portion. The base portion does not have any mechanism for moving the radiation generation apparatus. The base portion is a member for supporting the support pillar 4, and the support pillar 4 is erected vertically on the base portion. The leg portion 6 can be folded in a predetermined rotating direction with respect to the base portion.

The storage unit 10 for storing the radiation detector configured to detect radiation generated from the radiation generation unit 1 is attached to the support pillar 4. The storage unit 10 includes a hollow casing having an opening on one side, and an openable/closable cover installed in the opening of the casing. In other words, the storage unit 10 can accommodate an article to be stored such as the radiation detector because it is hollow inside. The control unit 11 configured to control exposure of the radiation generation unit 1 is also attached to the support pillar 4. The control unit 11 may be installed in the moving portion 8. The storage unit 10 and the control unit 11 are attached to the same side of the support pillar 4 as that of the handle 9. In other words, the storage unit 10 and the control unit 11 are detachably attached to the same side face of the support pillar 4.

The control unit 11 includes heavier components than the other units. By installing the control unit 11 at a lower end (near the moving portion 8 or the horizontal surface (floor surface or ground surface) of the support pillar 4, the weight balance of the radiation generation apparatus can be maintained.

At the support pillar 4, the storage unit 10 is attached above the control unit 11. This is because in a work flow during the photographing, a frequency of operator's accessing the radiation detector (storage unit 10) used for each photographing is higher than that of accessing the control unit 11. In the radiation generation apparatus according to the present exemplary embodiment, the control unit 11 is installed near the horizontal surface (floor surface or ground surface), and the storage unit 10 is installed, for example, at a position of 500 mm or more from the horizontal surface (floor surface or ground surface) where the operator can access the storage unit 10 without crouching. Therefore, the radiation detector can be easily taken out from the storage unit 10, and workability can be improved.

The control unit 11 may include a power source or a battery for supplying power to the radiation generation unit 1. The control unit 11 includes at least one of a control board for controlling exposure of the radiation generation unit 1, a power source, and a transformer for supplying power from the power source to the power board and the radiation generation unit 1. The radiation generation unit 1 and the control unit 11 are electrically connected to each other by wire or wireless.

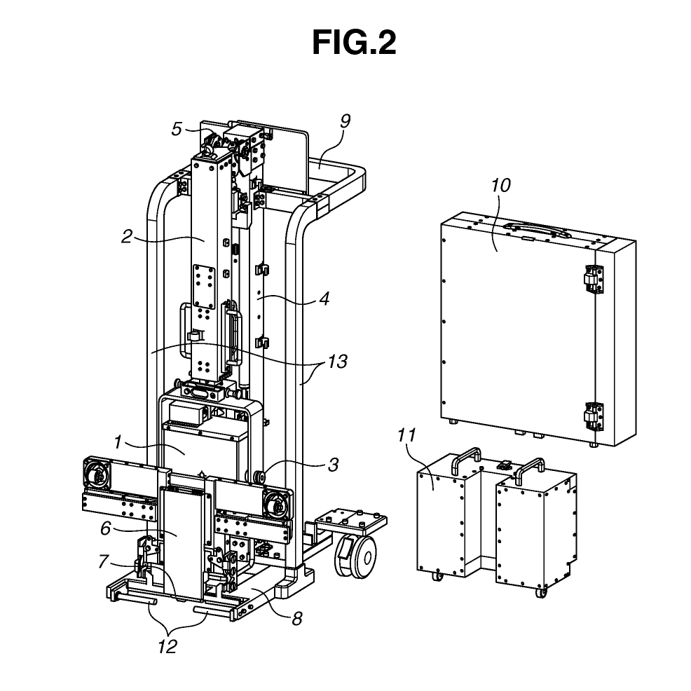

FIG. 2 illustrates a separated form where each of the storage unit 10 and the control unit 11 is removed from the radiation generation apparatus. This separated form is a transportation form for lifting and transporting the radiation generation apparatus.

By rotating the arm 2 in a predetermined rotating direction via the arm hinge portion 5, the arm 2 can be deformed from a form of expanding in an upper or horizontal direction illustrated in FIG. 1 to a form of being stored together with the radiation generation unit 1 illustrated in FIG. 2. When the arm 2 is stored together with the radiation generation unit 1 as illustrated in FIG. 2, the arm 2 is folded to be substantially parallel to the support pillar 4. In other words, the radiation generation unit 1 is disposed near the horizontal surface (floor surface or ground surface).

A grip portion 12 is a member secured to the moving portion 8 and gripped by the operator for lifting and transporting the radiation generation apparatus. As illustrated in FIG. 2, when the leg portion 6 is folded, the grip portion 12 to be gripped by the operator is exposed. In other words, when the leg portion 6 is folded, the grip portion 12 is exposed to a foremost front of the radiation generation apparatus.

The storage unit 10 for storing the radiation detector is detachable from the support pillar 4. The control unit 11 for controlling the exposure of the radiation generation unit 1 is similarly detachable from the support pillar 4. By removing the storage unit 10 from the support pillar 4, the radiation generation apparatus itself can be reduced in weight. Similarly, by removing the control unit 11 from the support pillar 4, the radiation generation apparatus itself can be reduced in weight. Thus, the operator can easily lift and carry the radiation generation apparatus.

The operator may remove either of the storage unit 10 or the control unit 11 from the support pillar 4. However, when only the control unit 11 is removed from the support pillar 4 without removing the storage unit 10, the center of gravity of the radiation generation apparatus is placed high, consequently the weight balance of the radiation generation apparatus is deteriorated. Accordingly, the operator first removes the storage unit 10 from the support pillar 4. Then, when further weight reduction of the radiation generation apparatus is necessary, the operator removes the control unit 11 from the support pillar 4. Thus, by presetting a removing order of objects from the support pillar 4 in this manner, the weight balance of the radiation generation apparatus can be maintained.

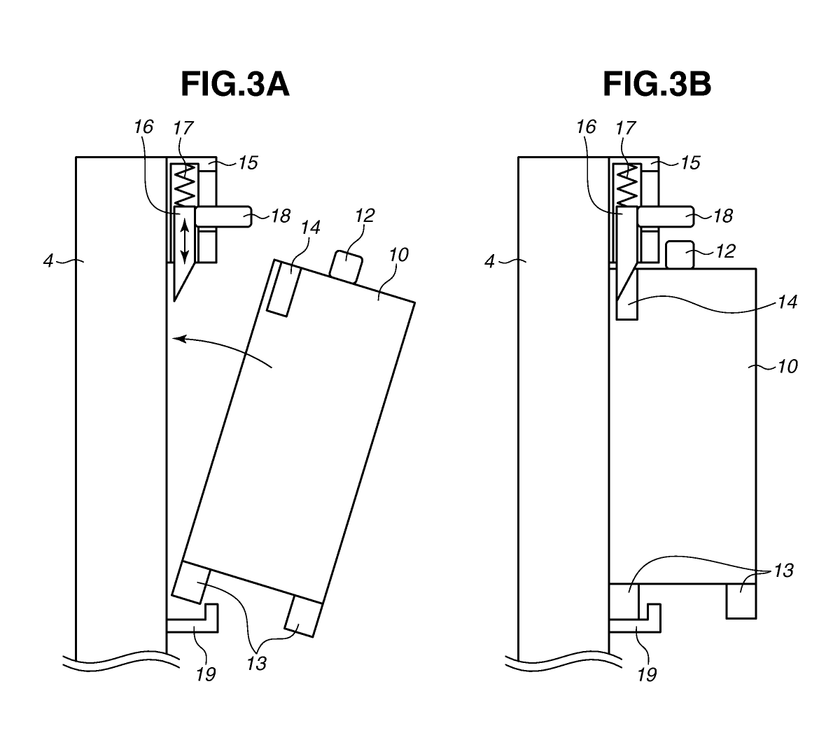

FIGS. 3A and 3B are diagrams each illustrating a detaching mechanism of the storage unit 10. FIGS. 3A and 3B are sectional views each illustrating a positional relationship between the support pillar 4 and the storage unit 10 when seen from a side face of the radiation generation apparatus. FIG. 3A illustrates a state where the storage unit 10 is detached from the support pillar 4. FIG. 3B illustrates a state where the storage unit 10 is attached to the support pillar 4.

As illustrated in FIGS. 3A and 3B, the storage unit 10 for storing the radiation detector includes the grip portion 12 to be gripped by the operator, a leg portion 13 for supporting the storage portion 10, and a groove 14 having space. The grip portion 12 is installed on a top surface of the storage unit 10, specifically, at a position where the operator can easily grip it without crouching. The leg portion 13 is installed on a bottom surface of the storage unit 10. A plurality of leg portions 13 is installed along the periphery of the bottom surface of the storage unit 10. Accordingly, the storage unit 10 can stand by itself. The groove 14 is formed on the top surface of the storage unit 10 nearer to the support pillar 4 side than the grip portion 12.

The support pillar 4 includes a lock portion 15 installed to lock the storage unit 10 so as to hold the storage unit 10 on the support pillar 4. The lock portion 15 may be integrated with the support pillar 4. The lock portion 15 includes a claw 16 to be fitted in the groove portion 14 formed in the storage unit 10 when the storage unit 10 is held on the support pillar 4, an force biasing portion 17 for biasing the claw 16 towards a locking position, and a mechanical pin 18 for releasing the locking position of the claw 16.

The claw 16 is formed of a hardened material and formed into a tapered shape. A leading (or tapered) end of the tapered shape of the claw 16 is arranged to engage with the groove portion 14 of the storage unit 10. The claw 16 is arranged parallel to the longitudinal direction of the support pillar 4 such that the tapered end becomes narrower as it descends downward parallel to the support pillar 4. The taper of the claw 16 is formed on a side opposite (not facing) a surface of the support pillar 4.

The force biasing portion 17 is installed within the casing of the lock portion 15 and is configured to apply tension to the claw 16 in the longitudinal direction of the support pillar 4. The force biasing portion 17 is, for example, a compression spring.

The pin 18 is a member secured to the claw 16 and configured to release the locking position (or locking force) of the claw 16. The claw 16 and the pin 18 are vertically movable within the casing of the lock portion 15. In other words, the claw 16 and the in 18 are mechanically interlocked.

The support pillar 4 includes a support portion 19 installed to engage and support a lower portion of the storage unit 10. Specifically, in one embodiment, the support portion 19 engages and supports at least one of the leg portions 13 of the storage unit 10.

When the storage unit 10 is attached to the support pillar 4, the operator holds the grip portion 12 of the storage unit 10 to lift the storage portion 10. Then, the operator places the leg portions 13 of the storage unit 10 on the support portion 19, and brings an upper portion of the storage unit 10 closer to the support pillar 4.

At this time, a side surface of the storage unit 10 abuts on the claw 16 to lift the claw 16 upward. Then, when the side surface of the storage unit 10 is contacted with the support pillar 4, the claw 16 is moved downward by the force biasing portion 17 to fit in the groove 14. The storage unit 10 is accordingly attached, and fitting thereof to the support pillar 4 is completed.

If necessary, the operator may move the pin 18 upward to lift the claw 16, and bring the upper portion of the storage unit 10 closer to the support pillar 4. Then, the operator may move the pin 18 downward so that the claw 16 moves downward to be fitted in the groove 14.

When the storage unit 10 is to be removed from the support pillar 4, the operator moves the pin 18 upward to overcome the resistance of the force biasing portion 17 and move the claw 16 upward. Since the claw 16 is pulled out from the groove 14, the fixing of the storage unit 10 is released. The operator holds the grip portion 12 of the storage unit 10 to tilt the storage unit 10 around the support portion 19, and then lifts the grip portion 12. Thus, the storage unit 10 can be removed from the support pillar 4.

Through the above-described operation, the storage unit 10 can be attached to/detached from the support pillar 4. When the radiation generation apparatus is moved via the moving portion 8, the operator can, by attaching the storage unit 10 to the support pillar 4, move the radiation generation apparatus and the storage unit 10 together.

When the radiation generation apparatus is lifted to be transported (carried), by removing the storage unit from the support pillar 4, the radiation generation apparatus and the storage unit 10 can be transported separately. In other words, when necessary, the radiation generation apparatus can be reduced in weight.

The lock portion 15 fixes the storage unit 10 by using the claw 16 or the force biasing portion 17. However, the storage unit 10 may be attached to the support pillar 4 by other methods, for example, by holding from both side faces of the storage unit 10 or by using magnetism. In other words, the lock portion 15 may be structured differently as long as it can removably afix the storage unit 10 to the support pillar 4 in the state where the storage unit 10 is attached to and can be removed form the support pillar 4.

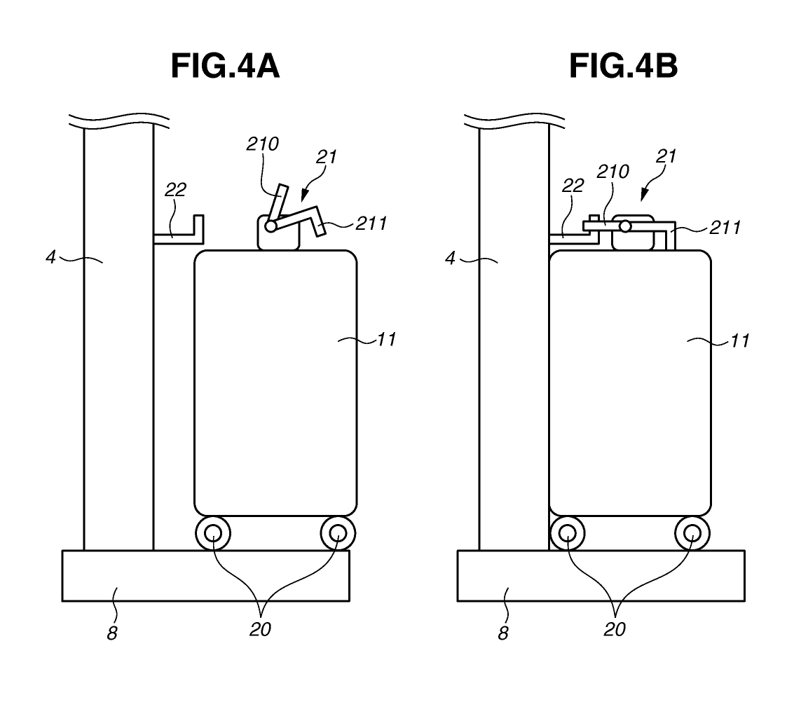

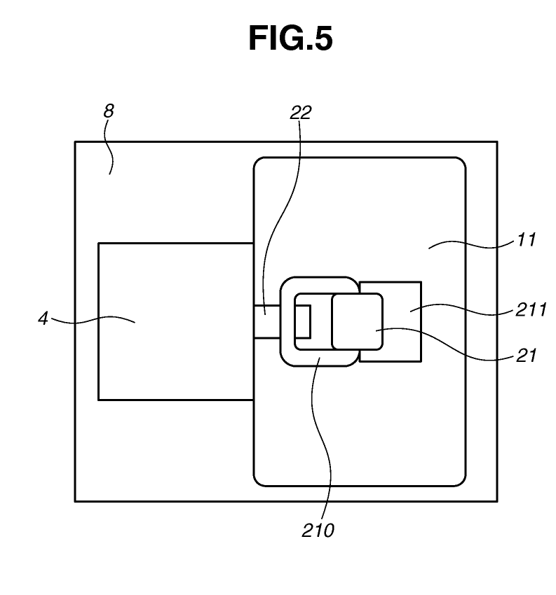

FIGS. 4A, 4B and 5 are diagrams each illustrating a detaching mechanism of the control unit 11. FIGS. 4A and 4B are side views each illustrating a positional relationship between the support pillar 4 and the control unit 11 seen from a lateral side of the radiation generation apparatus. FIGS. 4A and 4B illustrate a lower portion of the support pillar 4 engaged to the moving portion 8. FIG. 5 is a top view of the radiation generation apparatus illustrating a positional relationship between the support pillar 4 and the control unit 11 attached thereto, as seen from the upper side of the radiation generation apparatus.

FIG. 4A illustrates a state of the control unit 11 removed (disengaged) from the support pillar 4, while FIGS. 4B and 5 illustrate a state of the control unit 11 attached to the support pillar 4. As illustrated in FIG. 4A to 5, the mechanism for attaching and detaching the control unit 11 to the support pillar 4 is different from that for the storage unit 10.

The control unit 11 includes a plurality of wheels 20. Therefore, the control unit 11 can stand by itself using the plurality of wheels 20. This allow the operator to freely move the control unit 11 on the horizontal surface (floor surface or ground surface).

Further, the control unit 11 includes a connection portion 21 to connect the control unit 11 to the support pillar 4. As illustrated in FIGS. 4A to 5, the connection portion 21 includes, for example, an annular hooking portion 210 and an L-shaped lever 211. The L-shaped lever 211 and the hooking portion 210 are rotatably installed in the control unit 11. An angle of the hooking portion 210 can be changed by operating the L-shaped lever 211. For example, the hooking portion 210 can be lowered when the L-shaped lever 211 is pushed down, and can be lifted when the L-shaped lever 211 is raised.

The support pillar 4 includes a connected portion 22 connected to the connection portion 21. The connected portion 22 is, for example, a hook configured to mechanically engage with the hooking portion 210.

The connection portion 21 is installed on a top surface of the control unit 11. The connected portion 22 is installed on a side where the control unit 11 is installed (a side opposite to a side where the radiation generation unit 1, the arm 2, and the leg portion 6 are installed) on the support pillar 4. In a state where the control unit 11 is mounted on the moving portion 8 (base portion), the connected portion 22 is set at the same height as that of the connection portion 21.

When the control unit 11 is attached to the support pillar 4, the operator places the control unit 11 on the moving portion 8 (base portion). The operator moves, by rotating the wheels 20, the control unit 11 closer to the support pillar 4. Then, the operator hooks the connection portion 21 on the connected portion 22. Specifically, when the L-shaped lever 211 of the connection portion 21 falls over toward the top surface of the control unit 11, the hooking portion 210 falls to be hooked on the connected portion 22. Accordingly, tension is applied to the hooking portion 210 and the connected portion 22. Thus, the connection portion 21 is fixed to the connected portion 22. After the connection portion 21 is fixed to the connected portion 22, the attaching of the control unit 11 to the support pillar 4 is completed.

By raising the L-shaped lever 211 which has fallen over, the hooking portion 210 rises up. The connection portion 21 is disconnected from the connected portion 22, and the control unit 11 can be removed from the support pillar 4.

The wheel 20 of the control unit 10 near the support pillar 4 is installed nearer to the support pillar 4 than a contact point between the connection potion 21 and the connected portion 22. This is done for the purpose of maintaining weight balance of the control unit 11 attached to the support pillar 4.

Through the aforementioned operation, the control unit 11 can be attached to/detached from the support pillar 4. When the radiation generation apparatus is lifted to be carried, by removing the control unit 11 from the support pillar 4, the radiation generation apparatus and the control unit 11 can be separately moved. In other words, the radiation generation apparatus can be reduced in weight.

The radiation generation apparatus may have a limitation mechanism for limiting removal of the control unit 11 from the support pillar 4 unless the storage unit 10 is removed from the support pillar 4. The limitation mechanism is a mechanism for locking the control unit 11 to the support pillar 4 when the storage unit 10 and the control unit 11 are attached to the support pillar 4. Specifically, when the storage unit 10 and the control unit 11 are attached to the support pillar 4, the limitation mechanism limits rotation of the hooking potion 210 of the connection portion 21. The limitation mechanism releases, when the storage unit 10 is detached from the support pillar 4, the limitation of the rotation of the hooking potion 210 of the connection portion 21. Accordingly, the control unit 11 can be removed from the support pillar 4. Since the control unit 11 cannot be removed alone from the support pillar 4 without removing the storage unit 10 from the support pillar 4, the center of gravity of the radiation generation apparatus is not high. Therefore, the weight balance of the radiation generation apparatus can be maintained.

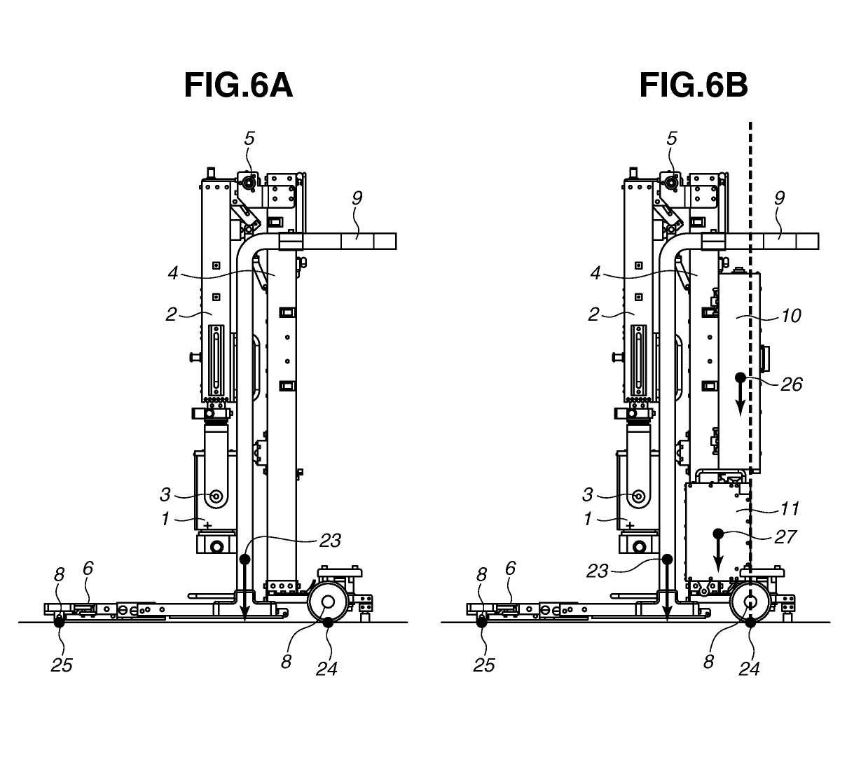

FIGS. 6A and 6B are diagrams each illustrating positions of centers of gravity of the storage unit 10 and the control unit 11 of the radiation generation apparatus. FIGS. 6A and 6B each illustrate an appearance of the radiation generation apparatus from a side face. FIG. 6A illustrates a state where the storage unit 10 and the control unit 11 are removed from the support pillar 4. FIG. 6B illustrates a state where the storage unit 10 and the control unit 11 are attached to the support pillar 4. FIG. 6A illustrates a symbol 23 indicating a position of the center of gravity and a weight vector of the radiation generation apparatus, a ground point 24 of the moving portion 8 in a rear portion of the radiation generation apparatus, and a ground point 25 of the moving portion 8 in a front portion of the radiation generation apparatus. Since the center of gravity of the radiation generation apparatus is between the front and rear ground points, tumbling of the radiation generation apparatus caused by external force in the back-and-forth direction is difficult to occur.

FIG. 6B illustrates a symbol 26 indicating a position of the center of gravity and a weight vector of the storage unit 10, and a symbol 27 indicating a position of the center of gravity and a weight vector of the control unit 11. As illustrated in FIG. 6B, the positions of the centers of gravity of the storage unit 10 and the control unit 11 are located before the ground point 24 of the moving portion 8 in the rear portion of the radiation generation apparatus. In other words, the positions of the centers of gravity of the storage unit 10 and the control unit 11 are between the front and rear ground points 24 and 25. Thus, irrespective of whether the storage unit 10 and the control unit 11 are detached, tumbling of the radiation generation apparatus caused by external force in the back-and-forth force direction of the radiation generation apparatus is difficult to occur.

As described above, the radiation generation apparatus according to the present exemplary embodiment includes the radiation generation unit 1 configured to generate radiation, the arm 2 configured to support the radiation generation unit 1, the support pillar 4 configured to support the arm 2, the moving portion 8 (base portion) configured to support the support pillar 4, and the storage unit 10 configured to store the radiation detector configured to detect the radiation. The storage unit 10 is detachable from the support pillar 4. The control unit 10 is also detachable from the support pillar 4. The arm 2 for supporting the radiation generation unit 1 may be detachable from the support pillar 4.

When lifting and carrying the radiation generation apparatus, by removing the storage unit 10 for storing the radiation detector from the radiation generation apparatus, the radiation generation apparatus can be reduced in weight. By removing the control unit 11 from the radiation generation apparatus, the radiation generation apparatus can be further reduced in weight.

When moving the radiation generation apparatus via the moving portion 8, the storage unit 10 for storing the radiation detector can be attached to the radiation generation apparatus. The control unit 11 can be also attached to the radiation generation apparatus. Therefore, since the radiation detector and the control unit 11 are integrated in the radiation generation apparatus, there is no more need to separately carry the radiation generation apparatus, the radiation detector, and the control unit 11.

Therefore, the components can be attached or detached according to the transportation form, and transportability can be improved.

Next, a second exemplary embodiment will be described. According to the second exemplary embodiment, a taking-out direction and a storing position of an article stored in a storage unit 10 are taken into consideration. The storage unit 10 is attached to a support pillar 4 so that the taking-out direction of the article (radiation detector) stored in the storage unit 10 is a horizontal direction and orthogonal to an advancing direction (moving direction or back-and-forth direction) of the radiation generation apparatus (moving portion 8).

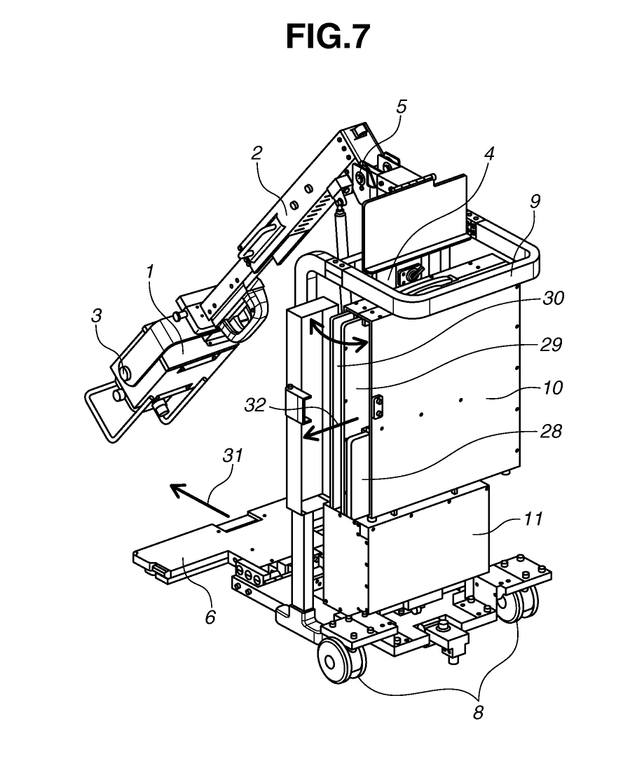

FIG. 7 is a perspective view illustrating a configuration of the radiation generation apparatus when seen from a rear side.

In FIG. 7, components 1 to 6, 8, 9, and 11 are similar to those described in the first exemplary embodiment. The storage unit 10 can store, together with a radiation detector 29, a personal computer (PC) 28 configured to set photographing conditions of the radiation generation unit 1, and a grid 30 configured to remove scattered rays of radiation. In other words, an article stored in the storage unit 10 is at least one of the PC 28, the radiation detector 29, and the grid 30. The storage unit 10 includes a holding portion for holding the PC 28, the radiation detector 29, and the grid 30 arranged in parallel. In the holding portion, the PC 28, the radiation detector 29, and the grid 30 are arranged at predetermined spaces so as not to contact one another. The holding portion is, for example, a plate-shaped projection installed according to a thickness of an article stored in the storage unit 10.

The PC 28 is a notebook or tablet type. The PC 28 can set patient information, and associate image data acquired from the radiation detector 29 with the patient information.

An arrow 31 indicates an advancing direction (moving direction or back-and-forth direction) of the radiation generation apparatus (moving portion 8). An arrow 32 indicates a taking-out direction of the article from the storage unit 10.

The storage unit 10 includes a hollow casing having an opening on one side, and an openable/closable cover installed in the opening of the casing. For example, the opening is formed on a side face of the storage unit 10 (casing) having the smallest area.

An operator can access the article stored in the storage unit 10 by opening the cover. Accordingly, the operator can take out the stored article through the opening of the storage unit 10. For example, the operator can take out the radiation detector 29 by sliding it in the taking-out direction 32 through the opening of the storage unit 10.

As illustrated in FIG. 7, the advancing direction (moving direction or back-and-forth direction) of the radiation generation apparatus (moving portion 8) is orthogonal to the taking-out direction 32 of the stored article. Since the advancing direction 31 is orthogonal to the taking-out direction 32, even when the stored article is taken out from the storage unit 10, movement of the radiation generation apparatus by the moving portion 8 can be suppressed. Therefore, since the stored article can be stably taken out from the storage unit 10, workability of the radiation generation apparatus can be improved.

Next, a specific configuration of the storage unit 10 will be described. In a state where the storage unit 10 is attached to the support pillar 4, when seen from a rear side of the radiation generation apparatus, the radiation detector 29 is disposed on a front side of the grid 30, and the PC 28 is disposed on a front side of the radiation detector 29. Specifically, the storage unit 10 holds the radiation detector 29 of a highest use frequency on a center of the storage unit 10, and the grid 30 of a use frequency lower than that of the radiation detector 29 on a deep side of the radiation detector 29. The storage unit 10 holds the PC 28 having an outer circumference smaller than that of the radiation detector 29 on the front side of the radiation detector 29. In other words, the storage unit 10 can hold the stored articles arranged in parallel according to the use frequencies or the sizes of the stored articles.

Specifically, when the radiation generation apparatus is moved in a hospital ward, the operator first takes out the PC 28 from the storage unit 10, and activates the PC 28. Accordingly, the storage unit 10 holds the PC 28 on the front most side. The operator checks information about a patient to be photographed next, by the PC 28. Then, when the radiation generation apparatus arrives at a place where the patient is present, the operator makes photographing preparation of the radiation generation apparatus. At this time, the operator takes out the radiation detector 29 from the storage unit 10. Depending on photographing, the grid 30 may not be used. A use frequency of the grid 30 is not higher than that of the radiation detector 29. Thus, the storage unit 10 holds the radiation detector 29 on the front side of the grid 30. In other words, the radiation detector 29 can be taken out more easily than the grid 30.

After photographing, the operator stores the radiation detector 29 or the like used for photographing in the storage unit 10, and turns off power for the PC 28 after an end of all photographing. Then, the operator stores the PC 28 in the storage unit 10. Thus, the photographing work flow described above can be efficiently carried out due to the internal configuration of the storage unit 10, and photographing work loads on the operator can be reduced.

As an outer configuration of the storage unit 10, a light material is preferred for the casing so as to improve portability. In view of exposure of the operator during the photographing, the storage unit 10 may be made of an exposure protection member for reducing transmission of radiation. By using a heavy metal, such as lead, molybdenum, or tungsten for a surface of the storage unit 10, which contacts the support pillar 4, or a surface opposite to the surface which contacts the support pillar 4, radiation transmission can be prevented while maintaining a thickness of a casing frame. With the aforementioned configuration, the operator hides behind the storage unit during the photographing, and exposure during the photographing can be reduced.

Next, a third exemplary embodiment will be described. According to the third exemplary embodiment, exposure of an operator is reduced without disposing any exposure protection member in a storage unit 10. A support pillar 4 includes an exposure protection member 33 for reducing transmission of radiation, and an unfolding portion 35 configured to unfold the exposure protection member 33 so as to cover the storage unit 10 when the storage unit 10 is attached to the support pillar 4.

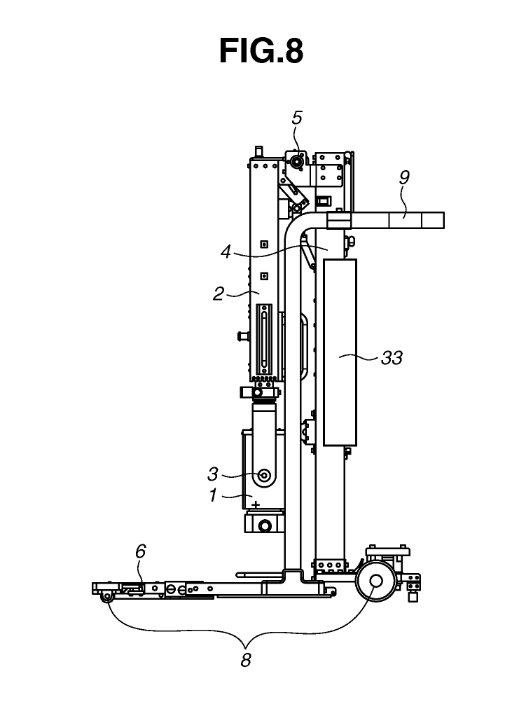

FIG. 8 is a diagram illustrating a configuration of a radiation generation apparatus according to the third exemplary embodiment, specifically an appearance of the radiation generation apparatus when viewed from a side face. In FIG. 8, components 1 to 6, 8, and 9 are similar to those described in the first exemplary embodiment. The exposure protection member 33 is installed in the support pillar 4. The exposure protection member 33 is unfolded when the storage unit 10 is attached to the support pillar 4.

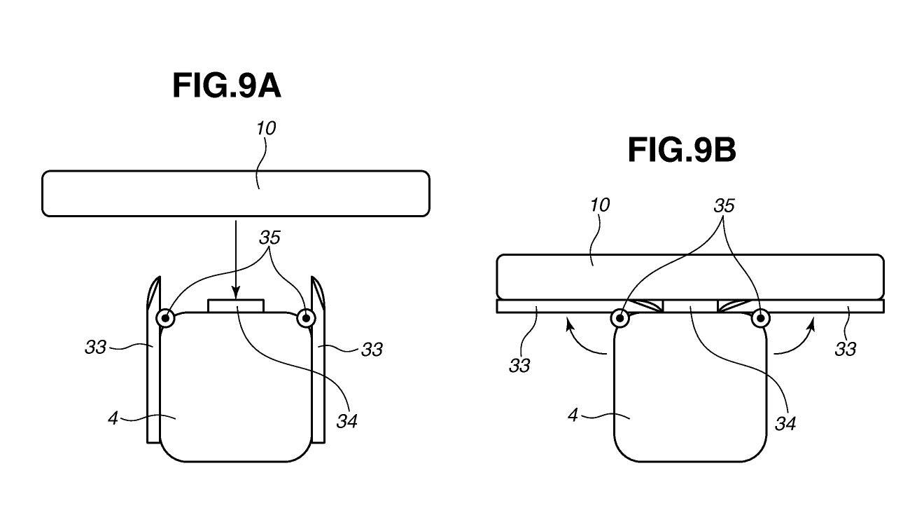

FIGS. 9A and 9B are sectional views illustrating the unfolding portion 35 configured to unfold the exposure protection member 33 and the support pillar 4 seen from a top surface of the radiation generation apparatus. FIG. 9A illustrates a state where the storage unit 10 is removed from the support pillar 4, and FIG. 9B illustrates a state where the storage unit 10 is attached to the support pillar 4.

As illustrated in FIGS. 9A and 9B, a second exposure protection member 34 is fixed to the support pillar 4. The unfolding portion 35 is, for example, a spring hinge. The unfolding portion 35 connects the support pillar 4 and the exposure protection member 33 between them. A rotational shaft of the unfolding portion 35 is set near a corner of the support pillar 4 in parallel to its longitudinal direction. Accordingly, the exposure protection member 33 is rotatable with respect to the support pillar 4. In a natural state where no external force is applied, the unfolding portion 35 applies a spring force so as to maintain the exposure protection member 33 in a form illustrated in FIG. 9A. The exposure protection member 33 has a tapered end formed in a circular arc shape on a side to which the storage unit 10 is attached as illustrated in FIG. 9A.

With this configuration, in the state where the storage unit 10 is removed from the support pillar 4, the exposure protection member 33 can be folded to the side face of the support pillar 4. Through the attaching operation of the storage unit 10 to the support pillar 4, the storage unit 10 comes into contact with a tapered leading end of the exposure protection member 33. As the storage unit 10 is closer to the support pillar 4, the exposure protection member 33 rotates more with respect to the support pillar 4. After the storage unit 10 is attached to the support pillar 4, the exposure protection member 33 completes its rotation and unfolds. To lock the storage unit 10 to the support pillar 4, the lock portion 15 of the first exemplary embodiment illustrated in FIG. 3 may be used.

Therefore, the radiation generation apparatus when the storage unit 10 is unnecessary can be miniaturized, and transportability can be improved. When the storage unit 10 is attached, the operator hides behind the storage unit during photographing, and exposure during the photographing can be reduced.

In the second exemplary embodiment, as the material for the exposure protection member of the storage unit 10, the heavy metal is used. According to the third exemplary embodiment, since limitation of arranging space and weight is eliminated due to the configuration of the storage unit 10, relatively inexpensive steel plates can be used as materials for the exposure protection members 33 and 34. As a result, according to the third exemplary embodiment, since no exposure protection member is used for the storage unit 10, the storage unit 10 can be reduced in weight.

A radiographic imaging system according to the present invention includes a radiation generation apparatus, a radiation detector configured to detect radiation generated by the radiation generation apparatus and passed through a subject and output image data according to the radiation, and a display configured to display the image data output from the radiation detector.

The radiation includes, in addition to α, β, γ, and X rays that are beams generated from particles (including photons) and emitted due to radioactive decay, a beam having energy of an equal or higher level, for example, a particle ray or a cosmic ray.

The arm 2 and the support pillar 4 of the radiation generation apparatus according to the present invention have been separately described. However, not limited to the arm 2 and the support pillar 4, one component having functions of the arm 2 and the support pillar 4 can be applied. This component is a member capable of connecting the radiation generation unit 1 and the moving portion 8 to each other and supporting the radiation generation unit 1. For example, the component has a bellows structure having predetermined rigidity, and is folded to store the radiation generation unit 1.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-095130 filed May 2, 2014, which is hereby incorporated by reference herein in its entirety.

What is claimed is:

1. A radiation generation apparatus comprising:
a radiation generation unit configured to generate radiation;
an arm configured to support the radiation generation unit;
a support pillar configured to support the arm;
a moving portion configured to support the support pillar and be movable; and
a storage unit is attachable to the support pillar and configured to store a radiation detector configured to detect the radiation,
wherein the storage unit is detachable from the support pillar.

2. The radiation generation apparatus according to claim 1, further comprising a control unit configured to control the radiation generation unit,
wherein the control unit is detachable from the support pillar.

3. The radiation generation apparatus according to claim 2, wherein the storage unit and the control unit are detachably attached to a same side surface of the support pillar.

4. The radiation generation apparatus according to claim 3, wherein in the support pillar, the storage unit is attached above the control unit.

5. The radiation generation apparatus according to claim 1, wherein a taking-out direction of the radiation detector stored in the storage unit is horizontal and orthogonal to a moving direction of the radiation generation apparatus.

6. The radiation generation apparatus according to claim 1, wherein the storage unit includes a hollow casing having an opening on one side thereof, and an openable/closable cover installed in the opening of the casing.

7. The radiation generation apparatus according to claim 1, wherein the storage unit includes a grip portion to be gripped by an operator and a leg portion configured to support the storage unit.

8. The radiation generation apparatus according to claim 1, wherein the storage unit stores, together with the radiation detector, a computer configured to set photographing conditions of the radiation generation unit, and a grid configured to remove scattered rays of the radiation.

9. The radiation generation apparatus according to claim 1, wherein the support pillar includes, to hold the storage unit on the support pillar, a lock portion installed to lock the storage unit.

10. The radiation generation apparatus according to claim 9, wherein the lock portion includes a claw configured to fit in a groove formed in the storage unit when the storage unit is attached to the support pillar, a force biasing portion configured to push the claw towards a locking position, and a pin configured to release the locking position of the claw.

11. The radiation generation apparatus according to claim 9, wherein the support pillar includes a support portion installed to support a lower portion of the storage unit.

12. The radiation generation apparatus according to claim 2, wherein the control unit includes a power source or a battery configured to supply power to the radiation generation unit.

13. The radiation generation apparatus according to claim 1, wherein the storage unit includes an exposure protection member configured to reduce transmission of the radiation.

14. The radiation generation apparatus according to claim 1, wherein the support pillar includes an exposure protection member configured to reduce transmission of the radiation and an unfolding portion configured to unfold the exposure protection member so as to cover the storage unit when the storage unit is attached to the support pillar.

15. A radiographic imaging system comprising:
the radiation generation apparatus according to claim 1;
a radiation detector configured to detect radiation passed through a subject and output image data according to the radiation; and
a display configured to display the image data output from the radiation detector.

16. A radiation generation apparatus comprising:
a radiation generation unit configured to generate radiation;
an arm configured to support the radiation generation unit;
a support pillar configured to support the arm;
a base portion configured to support the support pillar; and
a storage unit is attachable to the support pillar and configured to store a radiation detector which detects the radiation,
wherein the storage unit is detachable from the support pillar.
